(19) United States
(12) Patent Application Publication
(10) Pub. No.: US 2024/0398756 A1
(43) Pub. Date: Dec. 5, 2024

(54) DRUG FOR REDUCING BLOOD GLUCOSE AND/OR TREATING DIABETIC COMPLICATIONS

(71) Applicant: SICHUAN UNIVERSITY, Chengdu (CN)

(72) Inventors: Shiyong Zhang, Chengdu (CN); Xiao Xiao, Chengdu (CN); Xiaoluan Lu, Chengdu (CN); Yuhong Zhu, Chengdu (CN); Yulong Liao, Chengdu (CN); Chunyan Liao, Chengdu (CN); Xiao Wu, Chengdu (CN)

(21) Appl. No.: 18/804,173

(22) Filed: Aug. 14, 2024

Related U.S. Application Data
(63) Continuation of application No. PCT/CN2023/075700, filed on Feb. 13, 2023.

(30) Foreign Application Priority Data
Feb. 15, 2022 (CN) 202210137314.0

(51) Int. Cl. A61K 31/385; A61K 9/107; A61P 3/10

(52) U.S. Cl. CPC A61K 31/385; A61K 9/1075; A61P 3/10

(57) ABSTRACT

Disclosed is a drug for lowing blood glucose and/or improving diabetic complications, belonging to the technical field of biomedicine. According to the present disclosure, the drug for lowing blood glucose and/or improving diabetic complications is nanoparticles formed by a component containing lipoic acid and/or lipoic acid derivatives. The lipoic acid nanoparticles not only can control blood glucose safely and long-term, but also can realize the prevention/treatment of diabetic complications through lowering blood glucose and inhibiting oxidative stress. This drug has good clinical prospect.

### CROSS-REFERENCE TO RELATED APPLICATIONS

The present application is a continuation application of PCT application No. PCT/CN2023/075700 filed on Feb. 13, 2023, which claims the benefit of Chinese Patent Application No. 202210137314.0 filed on Feb. 15, 2022. The contents of all of the aforementioned applications are incorporated by reference herein in their entirety.

### TECHNICAL FIELD

The present disclosure belongs to the technical field of biomedicine, and particularly relates to a drug for reducing blood glucose and/or treating diabetic complications.

### BACKGROUND

Diabetes mellitus is a metabolic disorder characterized primarily by hyperglycemia, affecting up to 425 million patients worldwide. Metformin and acarbose, among other hypoglycemic drugs, are currently the conventional medications prescribed for diabetic patients in clinical practice. However, diabetic patients often suffer from complications such as cardiovascular diseases, obesity, nephropathy, retinopathy, and peripheral neuropathy. These medications, which solely focus on lowing blood glucose levels, have no therapeutic effect on these complications, resulting in the progression and deterioration of the condition in diabetic patients with comorbidities. Consequently, the development of hypoglycemic drugs with the function of improving complications is urgently needed.

At present, the main hypoglycemic drugs in clinical practice with the function of improving complications are glucagon-like peptide-1 receptor agonists (GLP-1RA) and sodium-glucose cotransporter 2 inhibitors (SGLT2i). They have demonstrated a certain degree of improvement in reducing the risks of cardiovascular mortality and end-stage renal disease mortality. Unfortunately, the range of indications of these two drugs is very limited, and further clinical research is needed to determine whether they can effectively treat complications. In addition, GLP-1RA requires daily subcutaneous injections, which can lead to poor patient compliance. Long-term use can also exacerbate the deterioration of diabetic retinopathy in patients. On the other hand, SGLT2i can increase the risk of urinary and genital infections, as well as gastrointestinal events. Therefore, hypoglycemic drugs that can improve diabetic complications are far from meeting the clinical needs.

### SUMMARY

To solve the above problems, the present disclosure provides a novel hypoglycemic drug and/or a drug for improving diabetic complications. This drug is nanoparticles mainly formed by a component containing lipoic acid and/or lipoic acid derivatives, could not only give a safe and long-lasting blood glucose control, but also effectively prevent and alleviate diabetic complications, and holds good clinical potential.

### The Present Disclosure is Realized Through the Following Technical Solutions

The present disclosure relates to lipoic acid nanoparticles with the functions of lowing blood glucose and/or improving diabetic complications. The lipoic acid nanoparticles are nanoparticles formed by a component containing lipoic acid and/or lipoic acid derivatives. The formation process may include various modifications of nanoparticles. The lipoic acid derivatives include lipoic acid salts or pharmaceutically acceptable modifiers (including but not limited to grafting functional groups on lipoic acid) obtained by non substantial modification that does not affect its core role.

As an optional method, in the aforementioned lipoic acid nanoparticles, the lipoic acid and/or lipoic acid derivatives are crosslinked and polymerized via disulfide bonds.

As an optional method, in the aforementioned lipoic acid nanoparticles, the nanoparticles can be formed by liposomes and polymers loading.

As an optional method, in the aforementioned lipoic acid nanoparticles, the nanoparticles can be formed by modification with polyethylene glycol.

As an optional method, in the aforementioned lipoic acid nanoparticles, the lipoic acid nanoparticles are made of small molecule lipoic acid monomers and/or lipoic acid derivatives through disulfide bonds crosslinking polymerization. Stable crosslinking can be achieved without the additional crosslinking molecules, and the composition is single and controllable. The crosslinked nanomedicine has a stable structure and is conducive to long-term circulation in the blood.

As an optional method, in the aforementioned lipoic acid nanoparticles, the hydrophilic and hydrophobic groups are outside and inside of the nanoparticles, respectively, which makes lipoic acid nanoparticles to dissolve in water without any cosolvent, and the solubility is much higher than that of lipoic acid monomer.

As an optional method, in the aforementioned lipoic acid nanoparticles, the particle size of the lipoic acid nanoparticles is 10-300 nm, far larger than the critical particle size through the capillary wall. Therefore, the lipoic acid nanoparticles can circulate to nerve tissues through blood circulation so as to extend greatly the retention time in the body, which enables effective absorption of lipoic acid in insulin-sensitive tissues and diabetic complications related tissues, leading to the improvement of therapeutic effect.

As an optional method, in the aforementioned lipoic acid nanoparticles, the lipoic acid nanoparticles show a surface negative potential, which is beneficial for improving the stability in the blood.

As an optional method, in the aforementioned lipoic acid nanoparticles, the surface potential of the lipoic acid nanoparticles is −100 mV-0 mV.

The present disclosure also provides a preparation method of the aforementioned lipoic acid nanoparticles, specifically, the disulfide bonds of the monomer and/or derivative of lipoic acid are crosslinked and polymerized to form lipoic acid nanoparticles through disulfide bond crosslinking polymerization reaction.

As an optional method, in the aforementioned preparation method of the lipoic acid nanoparticles, the methods of uniform mixing can be: ultrasonic oscillation, vortex oscillation, manual shaking, preferably ultrasonic oscillation.

As an optional method, in the aforementioned preparation method of the lipoic acid nanoparticles, the methods of breaking the disulfide bond of lipoic acid can be: ultraviolet light, ultrasound, heat, or mechanical stress.

As an optional method, in the aforementioned preparation method of the lipoic acid nanoparticles, the methods of cross-linking polymerization can be: oxygen supply, mechanical stress, or catalysis.

The present disclosure also provides an application of lipoic acid nanoparticles, wherein the lipoic acid nanoparticles are used for preparing a drug for lowing blood glucose and/or improving diabetic complications. As an optional method, in the aforementioned application, the lipoic acid nanoparticles are made into injection, capsule, tablet, pill, or oral liquid.

As an optional method, in the aforementioned application, the lipoic acid nanoparticles have good water-soluble property.

As an optional method, in the aforementioned application, the lipoic acid nanoparticles can be used in combination with other active components.

As an optional method, in the aforementioned application, the drug for improving diabetic complications can improve diabetic complications by lowing blood glucose and inhibiting oxidative stress.

As an optional method, in the aforementioned application, the mentioned diabetic complications comprise one or more of diabetic nephropathy, diabetic eye disease, diabetic foot disease, diabetic cardiovascular disease, diabetic cerebrovascular disease, and diabetic peripheral neuropathy.

The present disclosure also discloses a drug for lowing blood glucose and/or improving diabetic complications, wherein the drug contains lipoic acid nanoparticles.

As an optional method, other active ingredients are also included in the above drug for lowing blood glucose and/or improving diabetic complications. Furthermore, the other active ingredients can be other hypoglycemic drugs or other drugs with the function of improving diabetic complications, or other substances that can promote the hypoglycemia and/or improve the therapeutic efficacy of diabetic complications.

All features disclosed in this specification, or all methods or steps in the process, which could be combined in any way except mutually exclusive features and/or steps.

### The Beneficial Effect of the Present Disclosure

The nanodrug described in the present disclosure has long-lasting and efficient hypoglycemic effects, and its hypoglycemic effect and maintenance time are 1.53 and 3 times of the first-line clinical drug metformin, respectively. In addition, the nanodrug overcomes the defect that the existing hypoglycemic drugs lack efficacy for the treatment of complications, and shows the excellent therapeutic effects on multi-complications, holding a good clinical potential.

### BRIEF DESCRIPTION OF THE DRAWINGS

FIG. 1 shows a schematic diagram of lipoic acid nanoparticles in the treatment of diabetes and its complications;

FIG. 2 shows the hydrated particle size of lipoic acid nanoparticles after crosslinking;

FIG. 3 shows the Zeta potential of stable lipoic acid nanoparticles after crosslinking;

FIG. 4 shows the dilution stability determination of crosslinked lipoic acid nanoparticles;

FIG. 5 shows the fetal bovine serum (FBS) stability determination of crosslinked lipoic acid nanoparticles;

FIG. 6 shows the change of blood glucose, insulin content and insulin resistance index (HOMA-IR) for each group of experimental diabetic mice;

FIG. 7 shows the change of blood glucose for each group of experimental diabetic mice with nephropathy;

FIG. 8 shows the levels of UACR, Scr and BUN, and the representative images of H&E staining, PAS staining and podocytes TEM micrographs in experimental diabetic mice with nephropathy;

FIG. 9 shows the MDA content, SOD activity and the mRNA levels of TNF-α, IL-1β and IL-6 in kidney tissues of experimental diabetic mice with nephropathy;

FIG. 10 shows the change of the heat pain threshold of experimental diabetic mice with peripheral neuropathy;

FIG. 11 shows the change of the mechanical threshold of experimental diabetic mice with peripheral neuropathy;

FIG. 12 shows the change of the Na⁺—K⁺-ATPase activity in erythrocytes (a) and sciatic nerves (b) of experimental diabetic mice with peripheral neuropathy;

FIG. 13 shows the change of the content of MDA and GSH, and the activity of SOD in sciatic nerves of experimental diabetic mice with peripheral neuropathy;

FIG. 14 shows the content of TNF-α, IL-1β and IL-6 in sciatic nerves of experimental diabetic mice with peripheral neuropathy;

FIG. 15 shows the change of the morphological structure of sciatic nerves in experimental diabetic mice with peripheral neuropathy.

### DETAILED DESCRIPTION OF THE EMBODIMENTS

The above contents of the present disclosure are further explained in detail by the specific embodiments. However, this should not be construed as limiting the scope of the present disclosure to the following embodiments. Any modification without departing from the spirit and principles of the present disclosure, and any equivalent substitution or improvement based on the general technical knowledge and customary means in the art, shall be included in the scope of protection of the present disclosure.

### Embodiment 1

### 1. Preparation of Lipoic Acid Nanocesicles

210 mg of lipoic acid (LA) and 43.5 mg of template molecules 1, 4, 7-triazononane were dissolved in 3.5 mL of dimethyl sulfoxide (DMSO) and oscillated for 4 h to form a superamphiphilic solution. Under ultrasonic conditions, the above superamphiphilic solution was slowly added dropwise into 300 mL of deionized water to form the mother liquor of lipoic acid nanovesicles composed of LA and 1, 4, 7-triazononane. The mother liquor was exposed to 365 nm ultraviolet light for 4 h to induce self-crosslinking of the lipoic acid disulfide bond, NaOH adjusted the pH to about 9.0, dichloromethane was extracted 3 times to remove 1, 4, 7-triazononane in the solution, and then the supernatant was adjusted to neutral with dilute HCl, and then dialyzed with deionized water for 48 h (spectrum/pore), MWCO 2000) to obtain crosslinked lipoic acid nanovesicles.

### Size and Potential Detection of Crosslinked Lipoic Acid Nanoparticles:

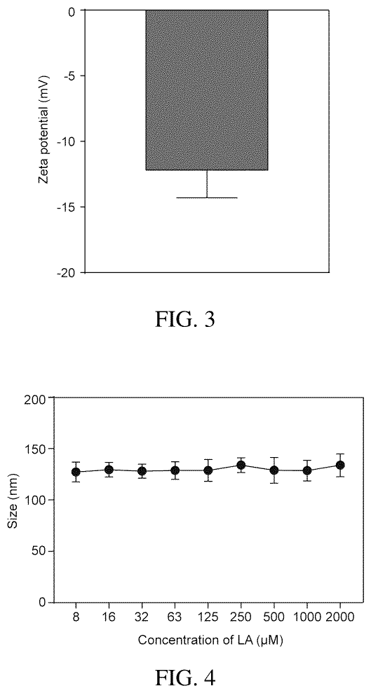

2 mL of the prepared crosslinked lipoic acid nanoparticles were placed into a 3.5 mL standard quartz dish, and their size and Zeta potential were determined by Dynamic Light Scatterometer (DLS). The results showed that the particle size of the crosslinked lipoic acid nanoparticles was about 130 nm, and the Zeta potential was −12.2 mV. As shown in FIGS. 2 and 3.

### Stability Detection of Crosslinked Lipoic Acid Nanoparticles:

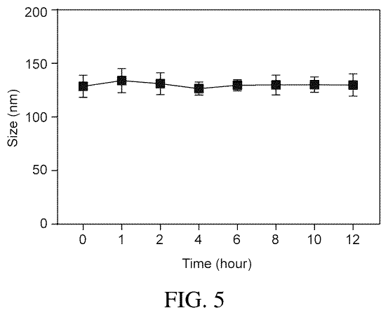

(1) Dilution stability: The crosslinked lipoic acid nanoparticle solution prepared above was taken and diluted to 2000/1000/500/250/125/63/32/16/8 μM (expressed as the concentration of LA) by RO water, and the particle size of the lipoic acid nanoparticles after each dilution was determined. As shown in FIG. 4, even when the solution of lipoic acid nanoparticles was diluted to 8 μM, the size remained close to the initial size, indicating that the prepared crosslinked lipoic acid nanoparticles had good dilution stability. (2) Serum stability: 4.5 mL of the above-mentioned crosslinked lipoic acid nanoparticles were incubated with 0.5 mL of fetal bovine serum, and the particle size of the nanoparticles was detected at different time points. As shown in FIG. 5, the size of the crosslinked lipoic acid nanoparticles did not change significantly during the 12 h of serum incubation, indicating that the prepared crosslinked lipoic acid nanoparticles had excellent serum stability. The above results show that the lipoic acid nanoparticles prepared by the method of the present disclosure have good stability, are conducive to long-term storage, allow large-scale preparation, and reduce the synthesis cost.

### Embodiment 2

### Preparation of Lipoic Acid Nanomicelles

300 mg of LA was added into 150 mL of deionized water, 1 M of NaOH aqueous solution was gradually added dropwise under stirring conditions until LA was completely dissolved, and then 1 M of HCl solution was used to titrate LA solution to neutral, and sodium lipoate powder was obtained after freeze-drying of the solution. 41.2 mg of sodium lipoate was weighed, dissolved in 1 mL of deionized water, and phacoemulsified to form lipoic acid nanoparticles. The obtained nanoparticles were exposed to 365 nm ultraviolet light to induce self-crosslinking of lipoic acid disulfide bond, reaction for 2.5 h, and after dialysis for 48 h, the crosslinked lipoic acid nanomicelles with a size of about 15 nm and a Zeta potential of about-33 mV were obtained.

### Embodiment 3

### Preparation of Lipoic Acid Nano-Aggregates

41.2 mg of lipoic acid was dissolved in 1 mL of N, N-dimethylformamide (DMF), and 0.2 M mother liquor of lipoic acid was obtained after oscillating on an oscillator for 2 h. 50 μL of the mother liquor was added into 5 mL of deionized water under ultrasonic conditions to obtain lipoic acid nanoparticles. The obtained nanoparticles were exposed to 365 nm ultraviolet light to induce self-crosslinking of lipoic acid disulfide bond, reaction for 2.5 h, and after dialysis for 48 h, the crosslinked lipoic acid aggregates with a size of about 80 nm and a Zeta potential of about −30 mV were obtained.

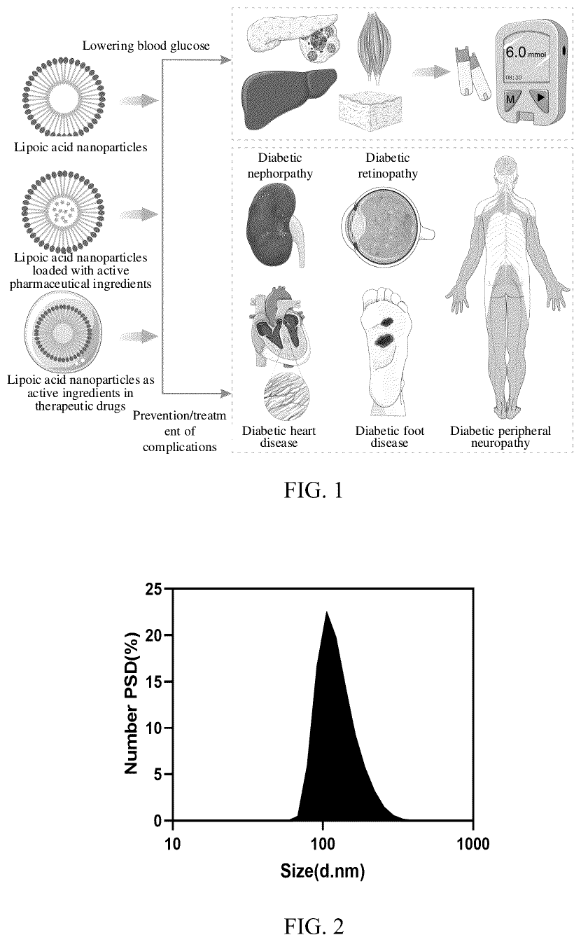

FIG. 1 shows a schematic diagram of lipoic acid nanoparticles in the treatment of diabetes and its complications.

### Embodiment 4

In this embodiment, db/db mice were used as a type 2 diabetes model to evaluate the efficacy of lipoic acid nanoparticles on diabetes mellitus, and compared with LA monomer and metformin hydrochloride, a first-line clinical hypoglycemic drug.

The lipoic acid nanoparticles prepared in Embodiment 1 were dispersed in normal saline to make a solution of 10 mg/mL, and mice were given intragastric treatment at a dose of 100 mg/kg.

### 1. Experimental Materials and Methods

### S1. Instruments and Reagents:

Blood Glucose Detector, Sinocare Inc.
Insulin, Shanghai Beyotime Biotech Inc.
Mouse Insulin ELISA assay kit, Jiangsu Jingmei Biotechnology Co., Ltd.

### S2. Experimental Materials and Animal Handling

8-week male C57BL/KsJ db/db mice, SPF grade, provided by Jiangsu GemPharmatech Co. Ltd., Changzhou Branch, certificate number: 202012449, with an initial weight of 44±2 g, underwent adaptive feeding for one week. A total of 24 C57BL/KsJ db/db mice were randomly divided into 4 groups according to body weight and blood glucose, with 6 mice in each group. They were db/db model group, db/db+lipoic acid nanoparticle group (100 mg/kg), db/db+lipoic acid monomer group (100 mg/kg) and db/db+metformin group (120 mg/kg). There were 6 littermate w/w male mice of the same week age as the solvent control group. Different doses of the drug were given by gavage according to the volume of 10 ml/kg, lipoic acid monomer and metformin once a day, the lipoic acid nanoparticle group once every three days for 1 month, and the solvent control group and the model group were given the same volume of saline by gavage. After administration of a single dose, the blood glucose changes of mice were continuously monitored for 3 days. During the 1-month treatment cycle, the blood glucose changes of the mice were detected every 3 days. On the 30th day, 2 h after the end of administration, the blood glucose of mice was measured, and the percentage of blood glucose reduced by the drug was calculated based on the difference between the blood glucose of the model and the normal control group. The mice were killed by enucleation and bloodletting, serum samples were collected by centrifugation at room temperature, the serum insulin content was detected by mouse insulin ELISA detection kit, and the insulin resistance index was calculated, and the percentage of the drug to reduce the serum insulin content and insulin resistance index was calculated based on the difference between the model and the normal control group.

### S3. Statistical Method

The results of the quantitative experiment were expressed as mean±variance (x̅±s). SPSS version19.0 Statistical Software (Chicago, IL, USA) was used to compare the differences between groups using one-way analysis of variance. When the difference was significant (P<0.05), the Turkey post hoc method was used for pairwise comparison, and when P<0.05, the difference was considered statistically significant.

### 2. Experimental Results

### S1. Effect of Lipoic Acid Nanoparticles on Blood Glucose in Experimental Diabetic Mice

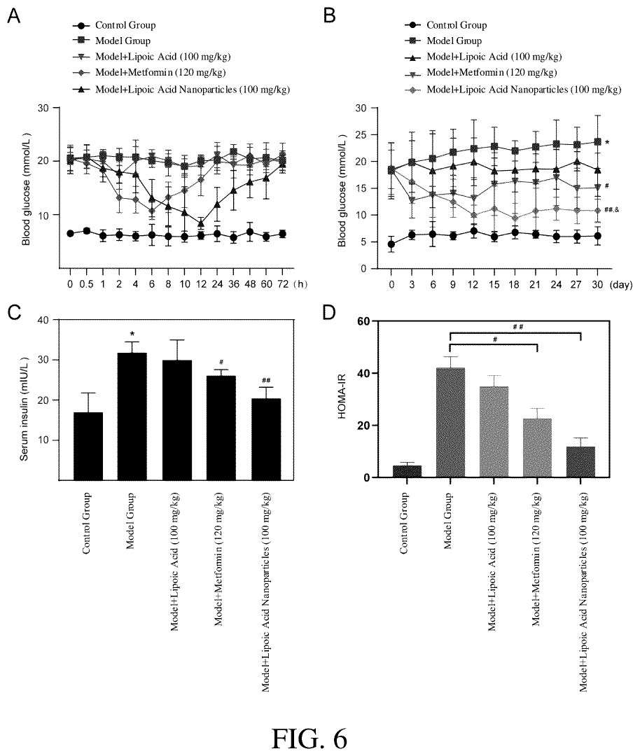

In order to investigate the therapeutic effect of lipoic acid nanoparticles on type 2 diabetes, we delivered lipoic acid monomer (100 mg/kg), lipoic acid nanoparticles (100 mg/kg) and metformin (120 mg/kg) into db/db mice by gavage, with solvent gavage administered to w/w mice as controls. The results of continuous glucose monitoring after a single dose showed that there was no significant difference in blood glucose levels between the LA monomer group and the model group (P>0.05), and the metformin group only showed a hypoglycemic effect within 24 h (P<0.05), while the lipoic acid nanoparticle group had a hypoglycemic effect of up to 72 h and a minimum blood glucose value of 8.4 mmol/L, which was 21.50% lower than that of metformin (A of FIG. 6). The experimental results showed that lipoic acid nanoparticles had a long-term and efficient effect on lowing blood glucose levels in diabetic mice.

During the one-month treatment period, the blood glucose test results are shown in B of FIG. 6. The LA monomer treatment group maintain blood glucose levels near the initial values of diabetic mice, with no hypoglycemic effect (P>0.05). Although the metformin group could reduce blood glucose contents by 50.28% (P<0.05), it required daily continuous administration. In contrast, lipoic acid nanoparticles administered once every 3 days, reduced blood glucose contents in mice by 76.84% (P<0.01), which was 1.53 times that of the metformin treatment group, indicating a significantly better hypoglycemic effect compared to the clinical first-line drug. Significant differences were analyzed between the control group and the db/db mouse group (*P<0.05). Significant differences were analyzed in the lipoic acid monomer group and the lipoicacid nanoparticle group, compared with the db/db mouse group (^{#}P<0.05, ^{##}P<0.01). Significant differences were analyzed between the lipoic acid nanoparticle group and the lipoic acid monomer group as well as the metformin group (^{&}P<0.05).

### S2. Effect of Lipoic Acid Nanoparticles on Serum Insulin Content and Sensitivity in Diabetic Mice

As shown in C and D of FIG. 6, the analysis of serum insulin contents and insulin resistance index is as follows. The serum insulin content and insulin resistance index of the db/db model group were significantly higher than those of the w/w control group (P<0.05), indicating that there was a serious effect of insulin resistance in the diabetic mice, and compared with the db/db model group, there was no significant difference in serum insulin content and insulin resistance index in the LA monomer group (P>0.05). Metformin, as an anti-diabetic clinical drug that reduces blood glucose by increasing the sensitivity of surrounding tissues to insulin, decreased the serum insulin content by 34.69% (P<0.05) and the insulin resistance index by 53.68% (P<0.05) after the end of the treatment cycle. It is worth noting that after the treatment of lipoic acid nanoparticles, the serum insulin content in diabetic mice decreased by 73.47% (P<0.01) and the insulin resistance index decreased by 79.84% (P<0.01), which were 2.1 and 1.5 times that of metformin, respectively (P<0.05). These results suggest that lipoic acid nanoparticles have a stronger insulin-sensitizing effect than first-line clinical drugs. Significant differences were analyzed in metformin group (120 mg/kg) and lipoic acid nanoparticle group (100 mg/kg), compared with db/db mouse group (*P<0.05, **P<0.01, ^{#}P<0.05, ^{##}P<0.01).

In the above embodiment, the vesicles in them are replaced with micelles and aggregates respectively, and similar experimental results are obtained, indicating that the various forms of lipoic acid nanoparticles prepared by the present disclosure all have the effect of lowing blood glucose levels for a long time and with high efficiency.

### Embodiment 5

### Experiment 1

10 mL of the aqueous solution of lipoic acid nanovesicles (10 mg/mL) prepared in Embodiment 1 was taken, and 8.3 mg of metformin hydrochloride was added into the solution. The lipoic acid nanoparticles/metformin physical mixture was obtained after mixing.

Referring to the method of preparing vesicles described in Embodiment 1, the difference is only that deionized water is replaced with an aqueous solution containing 25.0 mg of metformin hydrochloride, and lipoic acid nanovesicles loaded with metformin hydrochloride are prepared. Referring to the method described in Embodiment 4, the therapeutic effects of the physical mixture and the lipoic acid nanovesicles loaded with metformin hydrochloride in this Embodiment on diabetes mellitus were verified, respectively, and the results showed that: the hypoglycemic effect of the physical mixture was stronger than that of metformin hydrochloride and lipoic acid nanoparticles alone; and the therapeutic effect of lipoic acid nanovesicles loaded with metformin hydrochloride treatment increased further than that of the physical mixture in terms of lowering the active ingredient delivery while synergistically enhancing the therapeutic effect.

### Experiment 2

Referring to the method described in Experiment 1 of this Embodiment, replacing metformin hydrochloride therein with acarbose. The therapeutic effect of lipoic acid nanovesicles loaded with acarbose on diabetes was verified with reference to the method described in Embodiment 4. The results showed that the therapeutic effect of lipoic acid nanoparticless loaded with acarbose was significantly enhanced compared to acarbose and lipoic acid nanoparticless alone, which was basically consistent with the experimental conclusion of Experiment 1.

Lipoic acid nano-aggregates loaded with pioglitazone were prepared by taking 10 mL of an aqueous solution of lipoic acid nano-aggregates (10 mg/mL) prepared in Embodiment 3, adding 25 mg of pioglitazone to it, and dialyzing it for 48 h after mixing it sufficiently under ultrasonic conditions. The therapeutic effect of pioglitazone-loaded lipoic acid nano-aggregates on diabetes was verified with reference to the method described in Embodiment 4. The results showed that the therapeutic effect of lipoic acid nano-aggregates loaded with pioglitazone was significantly enhanced compared to pioglitazone and lipoic acid nanoparticles alone, which was basically consistent with the experimental findings of Experiment 1.

### Embodiment 6

This embodiment utilizes db/db mice as a model of diabetic nephropathy to evaluate the therapeutic effect of lipoic acid nanoparticles made in Embodiment 1 on mice with diabetic nephropathy in comparison to LA monomer.

### 1. Experimental Materials and Methods

### S1. Instruments and Reagents:

Blood glucose detector, Sinocare Inc.
Mouse albumin enzyme-linked immunization kit, Shanghai Tongwei Industrial Co., Ltd.
Creatinine (CRE) assay kit, Nanjing Jiancheng Bioengineering Institute.
Urea nitrogen (BUN) assay kit, Nanjing Jiancheng Bioengineering Institute.
Malondialdehyde (MDA) assay kit, Nanjing Jiancheng Bioengineering Institute.
Superoxide dismutase (SOD) assay kit, Solarbio science & technology (Beijing) Co., Ltd.
RNApure high purity total RNA fast extraction kit (centrifugal column type), Beijing BioTec Biotechnology Co., Ltd.
cDNA synthesis kit, Shanghai Beyotime Biotech Inc.
RT-PCR kit, Solarbio science & technology (Beijing) Co., Ltd.

### S2. Experimental Materials and Animal Treatment

10-week male C57BL/KsJ db/db mice, SPF grade, provided by Jiangsu GemPharmatech Co. Ltd., Changzhou Branch, certificate number: 202012449, with an initial weight of 44±2 g, underwent adaptive feeding for two weeks. They were randomly divided into 3 groups of 6 mice each according to body weight and blood glucose: db/db model group, db/db+lipoic acid nanoparticle group (30 mg/kg) and db/db+lipoic acid monomer group (30 mg/kg). There were 6 littermate w/w male mice of the same week age as the solvent control group. Different doses were given by intraperitoneal injection at 10 mL/kg volume once a day for four weeks, respectively, and equal volumes of normal saline were given by intraperitoneal injection in the control and model groups. During the treatment cycle, the blood glucose changes in mice were detected every three days, and the percentages of blood glucose reduced by the drugs were calculated based on the difference between the blood glucose of the model and the normal control group, so as to clarify the hypoglycemic effect of the drugs. At the end of the 4th week, 2 h after the administration of the drug, the blood glucose contents were detected, and the mice were killed by enucleation and bloodletting, blood samples were collected, and the serum specimens were separated by centrifugation for 15 min in a high-speed centrifuge at 3,000 rpm at room temperature; the serum contents of CRE and BUN were detected according to the instructions of the Creatinine (CER) and Urea Nitrogen (BUN) kits. At the same time, metabolic cages were used to collect urine samples from mice, and the albumin content in urine was detected by Enzyme-Linked Immunosorbent Assay (Elisa), and the urinary protein/creatinine rates (UACR) were calculated; mouse kidneys were dissected in sagittal plane, and ¼ of the tissues were taken and fixed by 4% paraformaldehyde, dehydrated, transparent, embedded in paraffin wax, and sliced, and the changes in the morphology and structure of the glomerulus were observed by Hematoxylin-Eosin (HE) staining, and the deposition of glycogen in glomeruli were detected by PAS staining method (Periodic Acid-Schiff stain); another 30 mg of tissue was taken to extract total RNA for detecting the transcript levels of inflammatory factors TNF-α, IL-6 and IL-1β genes; the remaining kidney tissues were processed by low-temperature homogenization, and then the tissues were detected according to the instructions of malondialdehyde (MDA) and superoxide dismutase (SOD) kits.

### MDA Content and SOD Activity.

### S3. Statistical Methods

The results of the quantitative experiment were expressed as mean±variance (x̅±s). SPSS version19.0 Statistical Software (Chicago, IL, USA) was used to compare the differences between groups using one-way analysis of variance. When the difference was significant (P<0.05), the Turkey post hoc method was used for pairwise comparison, and when P<0.05, the difference was considered statistically significant.

### 2. Experimental Results

### S1. Effect of Lipoic Acid Nanoparticles on Blood Glucose in Experimental Diabetic Mice

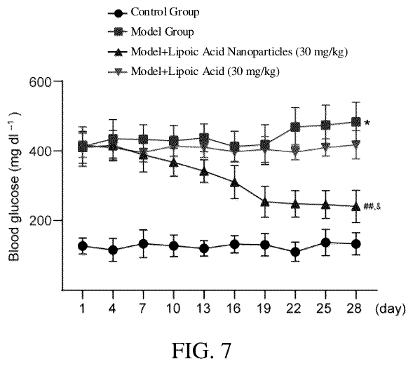

The results of blood glucose assay are shown in FIG. 7, the LA monomer group only stabilized the blood glucose near the initial blood glucose value of diabetic mice after treatment and had no hypoglycemic effect (P>0.05); whereas the same dose of lipoic acid nanoparticles effectively reduced the blood glucose content by 59.64% (P<0.01). The above results showed the beneficial effect of lipoic acid nanoparticles in improving glucose metabolism in diabetic nephropathy. Significant differences were analyzed between the control group and the db/db model group (*P<0.05). Significant differences were analyzed in lipoic acid monomer group and the lipoic acid nanoparticle group, compared with db/db model group (^{#}P<0.05, ^{##}P<0.01). Significant differences were analyzed between the lipoic acid nanoparticle group and the lipoic acid monomer group (^{&}P<0.05).

### S2. Effects of Lipoic Acid Nanoparticles on UACR, CRE, and BUN Contents and Kidney Tissue Pathology in Experimental Diabetic Mice

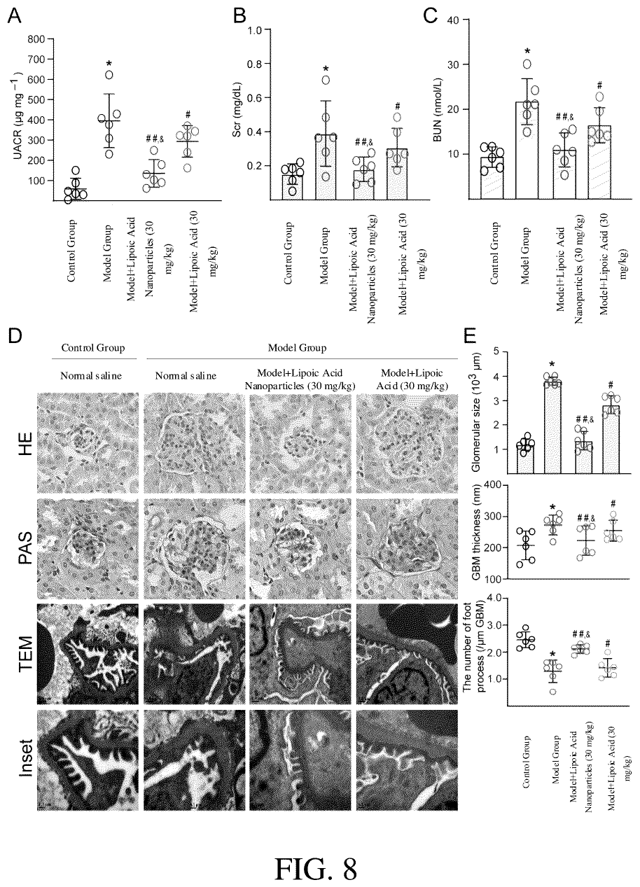

The results showed that compared with the db/db model group, serum CRE decreased by 21.05% (P<0.05), BUN content decreased by 25.45% (P<0.05), and UACR level decreased by 24.75% (P<0.05) in the LA monomer group, which indicated that LA monomer had an ameliorative effect on diabetic kidney injury. Furthermore, the lipoic acid nanoparticles group showed a 52.63% (P<0.01) reduction in serum CRE content, a 50.91% (P<0.01) reduction in BUN, and a 71.25% (P<0.05) reduction in UACR level (A-C of FIG. 8) compared with the db/db model group, which were 2.5, 2.0, and 3.3 times higher than the same dose of LA monomer, respectively. In addition, the pathological test results were shown in D and E of FIG. 8, the glomeruli of w/w control group mice had clear structure, normal basement membrane, and a small amount of glycogen deposition could be seen in the glomeruli, whereas the glomeruli of db/db model group mice were hypertrophied, with obvious thickening of the basement membrane, a large amount of glycogen deposition in the glomeruli, and a partial disappearance of podocyte foot processes, suggesting that there was severe glomerular injury, compared with the db/db model group, the glomeruli of LA monomer group shrank by 25.50% (P<0.05), the thickness of basement membrane decreased by 6.57% (P<0.05), and the number of foot processes increased by 10.14% (P<0.05). Whereas in the lipoic acid nanoparticles group, the glomeruli shrunk by 64.41% (P<0.01), the thickness of basement membrane decreased by 18.27% (P<0.01), and the number of foot processes increased by 64.17% (P<0.01), and the therapeutic effects were 2.5, 2.8 and 6.3 times higher than those of the same dose of LA monomer, respectively (P<0.05), demonstrating the excellent therapeutic effects of lipoic acid nanoparticles on diabetic renal impairment. Significant differences were analyzed in control group, the lipoic acid monomer group, and the lipoic acid nanoparticles group, compared with the db/db mouse group (*P<0.05, ^{#}P<0.05, ^{##}P<0.01). Significant differences were analyzed between the lipoic acid nanoparticles group and the lipoic acid monomer group (^{&}P<0.05).

### S3. Effects of Lipoic Acid Nanoparticles on the Gene Transcription Levels of MDA, SOD, and Inflammatory Factors TNF-α, IL-6, and IL-1β in the Kidney Tissue of Experimental Diabetic Mice

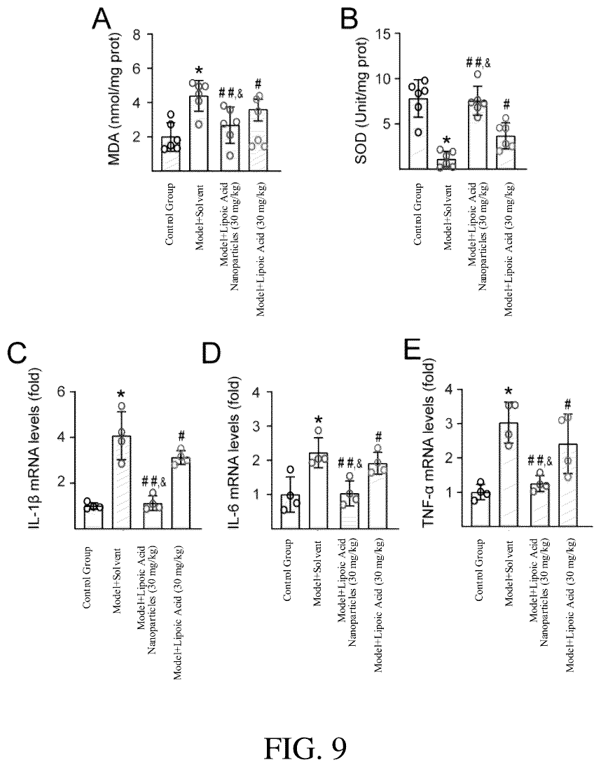

The improvement effect of diabetic renal oxidative damage was assessed by measuring MDA content and SOD activity in kidney tissues. The results showed that the increased MDA and decreased SOD activity levels in the kidneys of diabetic mice compared to control group mice indicated the presence of oxidative stress injury in the kidneys of db/db model mice. In addition, a significant increase in the transcript levels of inflammatory cytokine genes including TNF-α, IL-1β and IL-6 was observed in the kidney tissues of db/db diabetic mice. After 4 weeks of lipoic acid monomer treatment, MDA content was reduced by 9.8% (P<0.05) and SOD activity increased by 55% (P<0.05), indicating its ameliorative effect on renal oxidative stress injury. After treatment with lipoic acid nanoparticles, the MDA content in kidney tissues was significantly reduced by 46.34% (P<0.05), and the SOD activity was increased by 76.92% (P<0.05, A and B of FIG. 9), which were 4.7 and 1.4 times higher than that of the same dosage of LA monomer, respectively (P<0.05). In terms of inhibiting inflammatory responses, the TNF-α, IL-1β and IL-6 in the lipoic acid monomer group transcript levels were reduced by 22.5%, 18.18% and 23.33% (P<0.05), respectively, while the corresponding gene transcript levels in the lipoic acid nanoparticles group were reduced by 60%, 52.27% and 56.67% (P<0.01), respectively, which were 2.7, 2.9, and 2.4 times higher than those of the same dose of LA monomer (P<0.05, C-E of FIG. 9). The above results demonstrated the superior therapeutic effect of lipoic acid nanoparticles on oxidative stress and inflammatory injury in diabetic mouse kidney. Significant differences were analyzed in control group, lipoic acid monomer group, and lipoic acid nanoparticles group, compared with db/db model group (*P<0.05, ^{#}P<0.05, ^{##}P<0.01). Significant differences were analyzed between the lipoic acid nanoparticle group and the lipoic acid monomer group (^{&}P<0.05).

### Embodiment 7

21.0 mg of irbesartan, a therapeutic drug for diabetic nephropathy, was weighed and added into the aqueous solution of lipoic acid nanovesicles prepared in Embodiment 1 containing 100.0 mg. The lipoic acid nanovesicles loaded with irbesartan were prepared by dialysis for 48 h after being fully mixed under ultrasonic conditions. The therapeutic effect of the irbesartan-loaded lipoic acid nanodrug in this embodiment on diabetic nephropathy was verified by referring to the method described in Embodiment 6. The results showed that the efficacy of irbesartan-loaded lipoic acid nanoparticles was better than that of irbesartan monomer or lipoic acid nanovesicles. The irbesartan-loaded lipoic acid nanoparticles synergistically enhanced the therapeutic effect while reducing the dosage of active ingredients.

### Embodiment 8

In this embodiment, db/db mice were used as the model of diabetes peripheral neuropathy to evaluate the efficacy of lipoic acid nanoparticles prepared in Embodiment 1 on diabetes peripheral neuropathy (DPN), and compared with the clinical use of small molecule lipoic acid injection.

### 1. Experimental Materials and Methods

### S1. Instruments and Reagents:

Blood glucose detector, Sinocare Inc.
Photothermal tail pain meter, Chengdu TECHMAN Company.
RB-200 intelligent hot plate instrument, Chengdu TECHMAN Company.
Malondialdehyde (MDA) assay kit, Nanjing Jiancheng Bioengineering Institute.
Superoxide dismutase (SOD) assay kit, Nanjing Jiancheng Bioengineering Institute.
Glutathione (GSH) assay kit, Nanjing Jiancheng Bioengineering Institute.
Na⁺—K⁺-ATPase assay kit, Nanjing Jiancheng Bioengineering Institute.
Tumor necrosis factor-α (TNF-α) assay kit, Nanjing Jiancheng Bioengineering Institute.
Interleukin-6 (IL-6) assay kit, Nanjing Jiancheng Bioengineering Institute.
Interleukin-1β (IL-1β) assay kit, Nanjing Jiancheng Bioengineering Institute.

### S2. Animal Grouping and Experimental Materials

Eighteen 16-week male C57BL/KsJ db/db mice, SPF grade, provided by Jiangsu GemPharmatech Co. Ltd., Changzhou Branch. They were randomly divided into three groups according to body weight and blood glucose, with 6 mice in each group, namely the db/db model group, db/db+lipoic acid nanoparticles group (50 mg/kg) and db/db+lipoic acid injection group (50 mg/kg). The littermate w/w male mice of the same age were selected as the control group. The heat pain threshold was measured in all mice before administration to determine DPN symptoms. Lipoic acid injection and lipoic acid nanoparticles were intraperitoneally injected three times a week for 8 weeks. The control group and the db/db model group were intraperitoneally injected with an equal volume of saline. During the treatment period, blood glucose changes in mice were monitored every three days to determine the hypoglycemic effect of the drug. At the end of the 8th week, the heat pain threshold was detected. Mice were sacrificed to detect related factors through detection kits.

### S3. Thermal Pain Threshold Determination

The photothermal tail pain meter and the intelligent hot plate instrument were used to monitor pain stimuli and record the latency or threshold of tail flick and paw withdrawal after stimulation. In short, the corresponding parameters of the experimental device were set up, and the instrument was put into the mice after it was stable. When the paw retracted, struggled, or the tail swung, the value displayed by the instrument was recorded immediately and the mouse was released. Each mouse was evaluated three times at a 20-minute interval between two trials, and the mean value was used as the threshold.

S4. Na⁺—K⁺-ATPase Activity Determination

After 8 weeks treatment, all mice were sacrificed and the sciatic nerve was isolated. In order to determine the activity of Na⁺—K⁺-ATPase in red blood cells and sciatic nerve of mice, 10 μL whole blood was collected and added into 240 μL distilled water for mixing, and the activity of Na⁺—K⁺-ATPase was determined immediately. The sciatic nerve tissue of mice was collected. First, the sciatic nerve was cut into fragments (100 mg: 1800 μL) with small scissors in normal saline and homogenized with Beed Ruptor 24 Elite. Then, the homogenate was centrifuged at 4° C. and 3500 rpm for 15 min, and the supernatant was collected for use.

### S5. Measure of SOD Activity and MDA, GSH, TNF-α, IL-6 and IL-1β Contents in Sciatic Nerve

At the end of the experiment, mice were sacrificed and sciatic nerve cells were isolated. In order to determine the contents of MDA, GSH, SOD, TNF-α, IL-6 and IL-1β in the sciatic nerve, the sciatic nerve was first cut into fragments (100 mg: 1800 μL) with small scissors in normal saline and homogenized with Beed Ruptor 24 Elite. Then, the homogenate was centrifuged at 4° C. and 3500 rpm for 15 min, and the supernatant was collected for use. Then, commercial kits were used to determine the activity of SOD and the contents of MDA, GSH, TNF-α, IL-6 and IL-1β in sciatic nerve homogenate.

### S6. Morphological Analysis of the Sciatic Nerve

At the end of the experiment, the mice were sacrificed, and the sciatic nerve samples were isolated within 1-3 min. The size of the sample tissue was 2 mm×2 mm and try to be as thin as possible. During the sampling process, precise isolation of the intact sciatic nerve was ensured, avoiding mechanical damage such as compression by forceps, and a sharp blade was used to prevent tissue bruising. After the tissue was removed, it was immediately put into electron microscopy fixative at room temperature for 2 h of fixation, then transferred to 4° C. for storage. It was transported using an ice pack at 4° C., ensuring that the fixative remained in a liquid state during storage and transportation. Subsequently, sample preparation and analysis were performed.

### S7. Statistical Methods

The results of the quantitative experiment are expressed as mean±standard deviation (x±s). SPSS version19.0 Statistical Software (Chicago, IL, USA) was used to compare the differences between groups by one-way analysis of variance. When the difference was significant (P<0.05), the Turkey post hoc method was used for pairwise comparison between groups, and when P<0.05, the difference was considered statistically significant.

### 2. Experimental Results

### S1. Effects of Lipoic Acid Nanoparticles on Blood Glucose in Experimental Diabetes Peripheral Neuropathy Mice

The results of blood glucose test showed that the LA monomer group could only stabilize the blood glucose near the initial blood glucose value of diabetic mice after 8 weeks of treatment, and had no hypoglycemic effect (P>0.05), while the lipoic acid nanoparticles group effectively reduced the blood glucose content of diabetic mice, indicating that lipoic acid nanoparticles had a promoting effect on improving glucose metabolism in diabetic peripheral neuropathy.

### S2. Changes of Heat Pain Threshold in Mice of Each Group

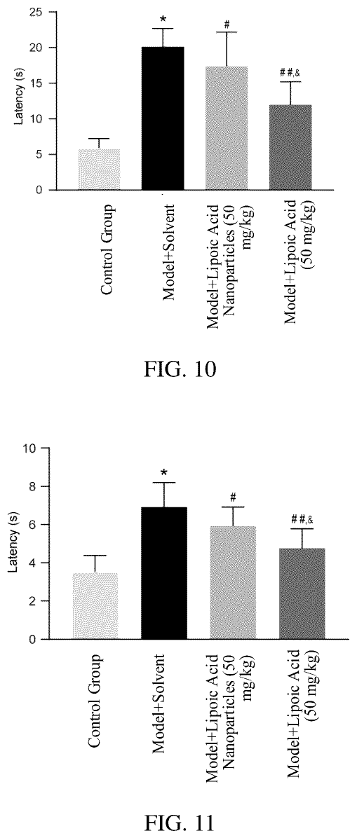

The results are shown in FIGS. 10 and 11, which are the hot plate pain threshold and the photoheat pain threshold, respectively. Compared with w/w mice, the latency of hot plate and tail-flick stimulation in db/db mice was prolonged (P<0.05), indicating that diabetic mice showed obvious pain retardation. Compared with db/db mice, the time of hot plate stimulation was shortened by 13.47% (P<0.05) and the time of tail-flick stimulation was shortened by 14.18% (P<0.05) in the lipoic acid injection group, indicating that LA monomer had a certain improvement effect on the behavior of diabetic peripheral neuropathy mice. Compared with db/db mice, the lipoic acid nanoparticles group showed a 40.43% (P<0.01) reduction in hot plate stimulation and a 31.13% (P<0.01) reduction in tail-flick stimulation, which were 3.0 and 2.2 times (P<0.05) of the same dose of lipoic acid injection, respectively. The above results showed that lipoic acid nanoparticles had excellent therapeutic effects in the behavioral aspects of diabetic peripheral neuropathy mice. Significant differences were analyzed between the control group and the db/db model group (*P<0.05). Significant differences were analyzed in lipoic acid monomer group and lipoic acid nanoparticle group, compared with db/db model group (^{#}P<0.05, ^{##}P<0.01). Significant differences were analyzed between the lipoic acid nanoparticles group and the lipoic acid monomer group (^{&}P<0.05).

S3. Changes of Na⁺—K⁺-ATPase Activity in Mice of Each Group

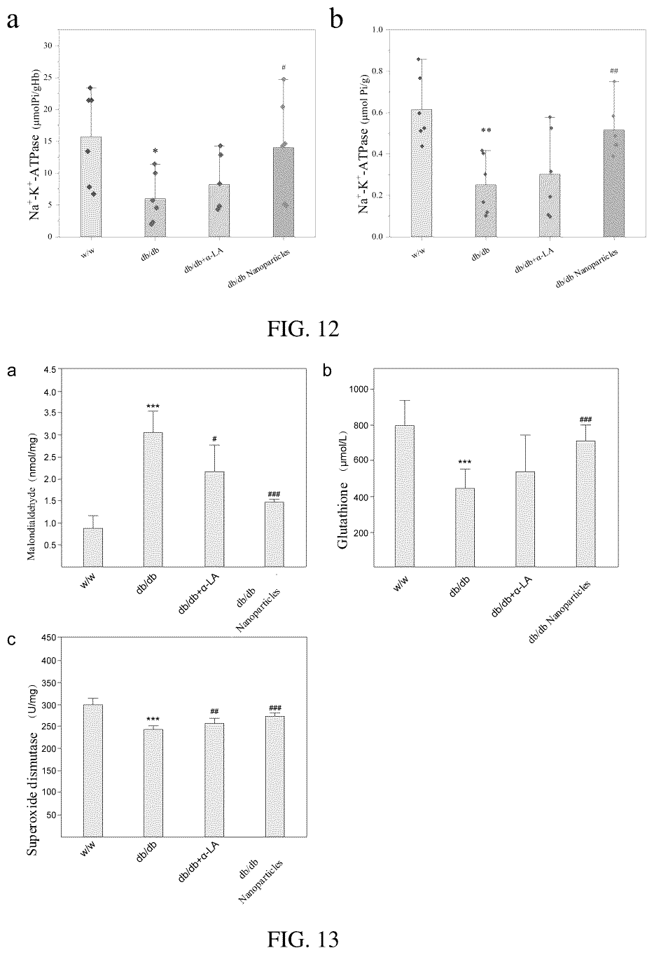

Na⁺—K⁺-ATPase activity is closely related to microvascular blood supply and peripheral nerve injury. The results are shown in FIG. 12, where a is serum Na⁺—K⁺-ATPase activity and b is sciatic nerve Na⁺—K⁺-ATPase activity. Compared with w/w mice, the activity of Na⁺—K⁺-ATPase in red blood cells and sciatic nerve of db/db mice was decreased. After 8 weeks treatment, compared with db/db mice, the Na⁺—K⁺-ATPase activity of red blood cells in the lipoic acid group increased by 137%, and the Na⁺—K⁺-ATPase activity in the sciatic nerve increased by 120%; compared with db/db mice, the erythrocyte Na⁺—K⁺-ATPase activity in the lipoic acid nanoparticle group increased by 234% (P<0.05). The activity of Na⁺—K⁺-ATPase in sciatic nerve was increased by 206% (P<0.01), which was 70.80% and 71.67% higher than that of lipoic acid monomer, respectively. The above results indicated that lipoic acid nanoparticles had a stronger therapeutic effect than clinical drugs in terms of restoring Na⁺—K⁺-ATPase activity in red blood cells and sciatic nerves of diabetic peripheral neuropathy mice. Significant differences were analyzed between w/w and db/db mice group (*P<0.05, **P<0.01, ***P<0.001). Significant differences were analyzed in lipoic acid injection group (50 mg/kg) and lipoic acid nanoparticles group (50 mg/kg), compared with db/db mice group (^{#}P<0.05, ^{##}P<0.01, ^{###}P<0.001).

### S4. Changes in SOD Activity and MDA, GSH, TNF-α, IL-6, and IL-1β Content in the Sciatic Nerve of Mice

As shown in FIG. 13, compared with w/w mice, the GSH content and SOD activity in the sciatic nerve of db/db mice decreased, and the MDA content increased significantly (P<0.01), indicating that there was obvious oxidative stress injury in the sciatic nerve of mice in the diabetic group. After 8 weeks of treatment, the MDA content in the lipoic acid group decreased by 30.79% (P<0.05), the SOD activity increased by 15.76% (P<0.05), and the GSH content increased by 10.79 (P<0.05). While in the lipoic acid nanoparticles group, the content of MDA decreased 51.75% (P<0.01), the activity of SOD increased 52.94% (P<0.01), and the content of GSH increased 17.72 (P<0.01), which were 1.7, 3.4 and 1.6 times of the same dose of LA monomer respectively (P<0.05). The above results showed that lipoic acid nanoparticles had excellent therapeutic effect on oxidative stress of sciatic nerve in mice with diabetic peripheral neuropathy. Significant differences were analyzed between w/w and db/db mice group (*P<0.05, **P<0.01, ***P<0.001). Significant differences were analyzed in lipoic acid injection group (50 mg/kg) and the lipoic acid nanoparticles group (50 mg/kg), compared with db/db mice group (^{#}P<0.05, ^{##}P<0.01, ^{###}P<0.001).

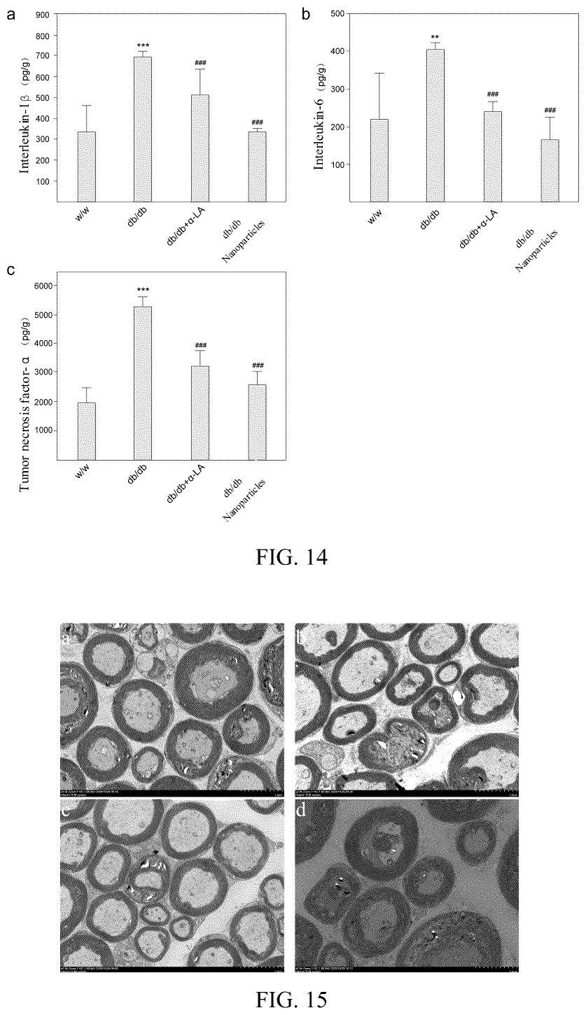

In terms of inhibiting inflammatory response, the results are shown in FIG. 14, compared with w/w mice, the levels of TNF-α, IL-1β and IL-6 cytokines in the sciatic nerve tissue of db/db mice were significantly increased, showing a stronger inflammatory response. Compared with db/db mice, the levels of TNF-α, IL-1β and IL-6 cytokines in the lipoic acid group were decreased by 34.42%, 27.46% and 38.42%, respectively (P<0.05), while the levels of corresponding cytokines in the lipoic acid nanoparticles group were decreased by 50.25%, 48.84% and 60.34%, respectively (P<0.01), which was 1.5, 1.8 and 1.6 times of the same dose of lipoic acid (P<0.05), revealing the excellent role of lipoic acid nanoparticles in the treatment of sciatic nerve inflammation in mice with diabetic peripheral neuropathy. Significant differences were analyzed between w/w and db/db mice group (*P<0.05, **P<0.01, ***P<0.001). Significant differences were analyzed in lipoic acid injection group (50 mg/kg) and the lipoic acid nanoparticles group (50 mg/kg), compared with the db/db mice group (^{#}P<0.05, ^{##}P<0.01, ^{###}P<0.001).

### S5. Changes of Sciatic Nerve Morphology in Mice of Each Group

db/db mice showed marked axonal demyelination and damage compared to w/w mice. Compared with db/db mice, lipoic acid injection group and the lipoic acid nanoparticles group showed significant remyelination after 8 weeks of treatment, and the effect of lipoic acid nanoparticles group was significantly better than that of the lipoic acid injection group, as shown in FIG. 15.

In FIG. 15, (a) is w/w mouse, (b) is db/db mouse, (c) is lipoic acid injection group (50 mg/kg), and (d) is lipoic acid nanoparticles group (50 mg/kg).

### Embodiment 9

This embodiment is lipoic acid nanoparticles tablet drug prepared using lipoic acid nanoparticles, comprising the following components expressed as mass fractions: 73% lipoic acid nanoparticles (main drug), 10% microcrystalline cellulose (filler), 10% starch slurry (binder), 6% corn starch (disintegrating agent), 0.2% magnesium stearate (lubricant), and 0.8% talc (lubricant).

The method of preparing the lipoic acid nanoparticle tablet drug is as follows:
Step 1. starting by sieving 73% of lipoic acid nanoparticles (main drug), 10% of microcrystalline cellulose (filler) and 6% of corn starch (disintegrating agent) through a 100-mesh sieve, respectively, followed by premixing these materials and sieving the mixture through a 20-mesh screen;
Step 2. adding 10% of starch slurry (binder) to prepare soft material, and passing the soft material through a 20-mesh sieve to form wet granules;
Step 3. drying the wet granules in a drying oven at 40° C. for 60 min, and then sieving the dried granules through a 20-mesh sieve for granulation;
Step 4. adding 0.2% of magnesium stearate (lubricant) and 0.8% of talc (lubricant) and mixing well;
Step 5. using a punch mold to press the tablet.

The db/db type 2 diabetic mice were utilized as a model of diabetic peripheral neuropathy to evaluate the application of tablet drugs prepared from lipoic acid nanoparticles and to compare their efficacy with the efficacy of commercially available small molecule lipoic acid tablets.

Eighteen 16-week male C57BL/KsJ db/db mice, SPF grade, were selected and provided by Jiangsu GemPharmatech Co. Ltd., Changzhou Branch. They were randomly divided into three groups of 6 mice each according to body weight and blood glucose, namely the db/db model group, the db/db+lipoic acid nanoparticles tablets group (50 mg/kg), and the db/db+small molecules lipoic acid tablets group (50 mg/kg). The same-week-old littermate w/w male mice were selected as the control group DPN symptoms were determined by measuring heat pain thresholds for all mice prior to drug administration. Small molecule lipoic acid tablets and lipoic acid nanoparticle tablets were administered orally three times a week, two tablets each time, for 8 weeks, and the blood glucose changes in mice were detected every 3 days during the treatment cycle to clarify the hypoglycemic effect of lipoic acid nanoparticles. At the end of the 8th week, the heat pain threshold was detected. Mice were sacrificed and other relevant factors were detected using assay kit. The results showed that the lipoic acid nanoparticles tablets were significantly better than the small molecule lipoic acid tablets in lowering blood glucose and heat pain threshold, elevating Na⁺—K⁺-ATPase activity, improving tissue lesions, and alleviating inflammation after 8 weeks of treatment, demonstrating the excellent therapeutic efficacy of the lipoic acid nanoparticles tablets obtained by the present embodiment for diabetic peripheral neuropathy.

The present disclosure has systematically sorted out the problems existing when the existing small molecule lipoic acid is used in the treatment of diabetes mellitus and its complications, and the research found that the existing small molecule lipoic acid mainly has the following major problems.

1. When small molecule lipoic acid is intravenously injected into the human body, it undergoes metabolism by the kidneys as it circulates in the bloodstream. 80-90% of the small molecule lipoic acid passes through the glomerulus into the renal tubules, where it is excreted in the urine. As a result, it is unable to reach some of the nerve tissues, has a short retention time, and exhibits a short duration of action, leading to poor therapeutic effects.

2. During the storage of small molecule lipoic acid, the disulfide bond in its structure is unstable. When exposed to heat or light, the disulfide bond in the dithiomole ring can break, forming unstable free radicals that lead to the inactivation of lipoic acid. In environments with low pH and high humidity, the sulfhydryl groups formed by the breakage of disulfide bonds can aggregate randomly, forming a number of obvious pale yellow gelatinous substances. These gelatinous substances are degradation products of lipoic acid polymers, which are difficult to dissolve and absorb. Therefore, the current small molecule lipoic acid has a short shelf life and requires stringent preservation conditions.

3. Due to the low solubility of lipoic acid in aqueous solution, the existing small molecule lipoic acid injection is prepared by adding alkaline co-solvents such as meglumine, ethylenediamine, and tromethamine to increase the solubility of small molecule lipoic acid in water, which not only reduces the content of lipoic acid in the injection per unit molarity, but also raises the risk of the injection of undesirable effects due to its complex composition. Moreover, alkaline co-solvents increase the potential for degradation of small molecule lipoic acid.

As for the lipoic acid nanoparticles in the drug for lowering blood glucose and/or improving diabetic complications described in the present disclosure, its particle size is 10-300 nm, which is much larger than the critical particle size of the glomerular filtration system, so that the lipoic acid nanoparticles can circulate in the body along with the blood to the nerve tissues of various parts of the body, greatly enhancing the retention time in the body, so that the peripheral nerve tissues can effectively absorb the lipoic acid, thereby enhancing the therapeutic effect. The disulfide bonds of the monomers are crosslinked and polymerized together, and the more active disulfide bonds are wrapped inside the nanoparticles, thus enhancing their stability and prolonging their shelf life.

The hydrophilic groups in the lipoic acid nanoparticles are outside the nanoparticles and the hydrophobic groups are inside the nanoparticles, which makes the lipoic acid nanoparticles soluble in water without any co-solvents, and the solubility is much higher than that of the lipoic acid monomer. In addition, the increased solubility without the addition of co-solvents reduces the cost and injection risk.

### Embodiment 10

Lipoic acid nanoparticles-loaded PLGA micelles were prepared by taking 25.0 mg of the lipoic acid nano-aggregates prepared in Embodiment 3 and adding them into an aqueous solution containing 100 mg of Poly(lactic-co-glycolic acid) (PLGA), which was thoroughly mixed and dialyzed under ultrasonication. The lipoic acid nanoparticles-loaded PDLLA micelles were prepared by taking 25.0 mg of the lipoic acid nano-aggregates prepared in Embodiment 3 and adding them into an aqueous solution containing 100 mg of Poly(D,L-lactide) (PDLLA), which was thoroughly mixed and dialyzed under ultrasonication. The hypoglycemic effect of above two nanoparticles obtained in this embodiment was verified with reference to the method described in Embodiment 4. The results showed that with different carrier loading, the removal time of the lipoic acid nanoparticles was prolonged and the time for the drug to exert its efficacy was increased, presenting a superior therapeutic effect.

### Embodiment 11

Referring to the method described in Embodiment 1, lipoic acid nanoparticles were prepared, and then polyethylene glycol (PEG-1000) was grafted onto the above lipoic acid nanoparticles by esterification with 1-(3-dimethylaminopropyl)-3-ethylcarbodiimide and 4-dimethylaminopyridine. Compared with the ungrafted nanoparticles, the surface modification of the lipoic acid nanoparticles with PEG-1000 effectively reduces the adsorption of the nanoparticles with proteins in the body, delays the removal of the nanoparticles, prolongs the time for the drug to exert its efficacy, and exhibits a superior therapeutic effect.

The above are only preferred embodiments of the present disclosure, and are only illustrative, not restrictive, of the present disclosure; and the person of ordinary skill in the art understands that many changes, modifications, or even equivalent changes can be made within the spirit and scope of the claims of the present disclosure, but all will fall within the scope of protection of the present disclosure.

What is claimed is:

1. An application of lipoic acid nanoparticles, wherein the lipoic acid nanoparticles are used for preparing a drug for lowing blood glucose and/or improving diabetic complications.

2. The application according to claim 1, wherein the lipoic acid nanoparticles are nanoparticles formed by a component containing lipoic acid and/or lipoic acid derivatives.

3. The application according to claim 1, wherein the drug for improving diabetic complications is arranged by lowing blood glucose and inhibiting oxidative stress.

4. The application according to claim 1, wherein the diabetic complications comprise one or more of diabetic nephropathy, diabetic eye disease, diabetic foot disease, diabetic cardiovascular disease, diabetic cerebrovascular disease, and diabetic peripheral neuropathy.

5. The application according to claim 1, wherein the lipoic acid nanoparticles combine with the other active ingredients.

6. Lipoic acid nanoparticles suitable for the application according to claim 1, wherein the lipoic acid nanoparticles are nanoparticles formed by a component containing lipoic acid and/or lipoic acid derivatives.

7. The lipoic acid nanoparticles according to claim 6, wherein the lipoic acid and/or the lipoic acid derivatives are crosslinked and/or polymerized via disulfide bonds.

8. The lipoic acid nanoparticles according to claim 6, wherein a particle size of the lipoic acid nanoparticles is 10-300 nm.

9. A drug for lowing blood glucose and/or improving diabetic complications, wherein the drug contains lipoic acid nanoparticles.

10. The drug for lowing blood glucose and/or improving diabetic complications according to claim 9, wherein the drug contains other active ingredients.
